(19) United States
(12) Patent Application Publication
(10) Pub. No.: US 2023/0114405 A1
(43) Pub. Date: Apr. 13, 2023

(54) Microemulsion Delivery Systems for A Female Hormone Blend with Alcohol-Soluble Species Including Nonderivatized Hormones

(71) Applicant: Quicksilver Scientific, Inc., Louisville, CO (US)

(72) Inventors: Christopher W. Shade, Louisville, CO (US); Steven Tieu, Louisville, CO (US)

(73) Assignee: Quicksilver Scientific, Inc., Louisville, CO (US)

(21) Appl. No.: 17/967,267

(22) Filed: Oct. 17, 2022

Related U.S. Application Data
(63) Continuation-in-part of application No. 17/672,894, filed on Feb. 16, 2022.
Application No. 17/672,894 is a continuation of application No. PCT/US20/49442, filed on Sep. 4, 2020.
(60) Provisional application No. 62/896,815, filed on Sep. 6, 2019.
(60) Provisional application No. 63/272,358, filed on Oct. 27, 2021.

(51) Int. Cl. A61K 31/568; A61K 47/14; A61K 47/44; A61K 9/00; A61K 9/107; A61K 9/48; A61P 5/26

(52) U.S. Cl. CPC A61K 31/568; A61K 47/14; A61K 47/44; A61K 9/0053; A61K 9/1075; A61K 9/4858; A61P 5/26

(57) ABSTRACT

Microemulsions are described where hydrophobic liquid droplets are distributed in a continuous hydrophilic liquid phase. The described microemulsions may be thought of as modified oil-in-water (MOIW) microemulsions, where both the “oil” and “water” phases of the microemulsion are modified. The oil phase droplets of the MOIW microemulsion are modified with alcohol and can solubilize alcohol-soluble species, including nonderivatized hormones. The polar continuous “water” phase of the MOIW microemulsion is modified with a sugar or sugar alcohol.

### REFERENCE TO RELATED APPLICATIONS

This application is a continuation-in-part of U.S. Nonprovisional application Ser. No. 17/672,894, filed Feb. 16, 2022, entitled “Microemulsion Delivery Systems for Alcohol-Soluble Species Including Nonderivatized Hormones,” which is a continuation of International Application No. PCT/US20/49442, entitled “Microemulsion Delivery Systems for Alcohol-Soluble Species Including Nonderivatized Hormones”, filed Sep. 4, 2020, which claims the benefit of U.S. Provisional Application No. 62/896,815 entitled “Microemulsion Delivery Systems for Alcohol-Soluble Species Including Nonderivatized Hormones” filed Sep. 6, 2019; and also claims the benefit of U.S. Provisional Application No. 63/272,358 entitled “Microemulsion Delivery Systems for An Adaptogenic Female Hormone Blend with Alcohol-Soluble Species Including Nonderivatized Hormones” filed Oct. 27, 2021, the contents of all are incorporated herein by reference in their entirety, except that in the event of any inconsistent disclosure or definition from the present application, the disclosure or definition herein shall prevail.

### BACKGROUND

Hormone replacement therapy (HRT) is used extensively for treating hormone deficiencies due to aging or pathological effects on the endocrine system. HRT also may be used to change secondary sexual characteristics of transgender people. Commonly used HRT hormones to combat menopause or andropause include testosterone, DiHydroTestosterone (DHT), DeHydroEpiAndrosterone (DHEA), 7-keto DHEA, progesterone, pregnenolone, estrogen, estradiol, estriol, androstenedione, and androstenediol.

Other than by injection or implant, the delivery of nonderivatized hormones to mammalian organisms can be difficult or unwise from a liver toxicity perspective. If delivered orally, conventional delivery systems often result in extensive metabolism of nonderivatized hormone in the liver, which may modify or render the hormone ineffective, and cause undesirable liver stress.

For example, oral delivery of nonderivatized testosterone results in negligible blood concentration of testosterone, as substantially complete “digestion” of the hormone occurs in the stomach and liver—putting stress on the liver. In contrast to testosterone, DHEA and progesterone can be taken orally in solid or suspended form, and if enough is taken orally in solid or suspended form, achieve effective bloodstream concentrations. However, ingesting either of these nonderivatized hormones in solid or suspended form places significant stress on the liver as the majority of these nonderivatized hormones are digested and not beneficially transferred to the bloodstream. Thus, oral delivery to achieve therapeutically effective bloodstream concentrations is practically non-existent for some nonderivatized hormones, while for others oral delivery results in substantial loss of the hormone—and in either case, undesirable stress is placed on the liver at a minimum, with liver damage being possible. This situation is especially apparent for nonderivatized hormones that are not well-solubilized in water or oil.

Nonderivatized hormone including transdermal creams and gels applied to various locations on the skin, including the underarm and nasal tissues, have been attempted to bypass liver metabolism. However, transdermal creams and gels often suffer limitations from poor and variable rates of absorption, especially over time, and the potential alteration of the hormones during transport through the skin by enzymes in the skin. Furthermore, being applied to the skin, such preparations are often transferred to clothing and other surfaces and can become a danger to other family members.

More recently nonderivatized hormone including solid pellets have been implanted under the skin. The pellets are designed to dissolve in body fluids over time, thus providing a somewhat continuous hormone dose over a 3- to 6-month period. While surgical implantation of the solid pellets is required, injection or daily transdermal application of the hormone is avoided. However, in practice the release of the hormone is often dependent on implant depth, tissue location of the solid pellets, and whether the pellets are undesirably agitated by impact or exercise. In combination, these additional variables, especially undesirable agitation arising from exercise, result in wide variance in the release profile of the hormone—commonly in the form of initial phase of over-dose and a later phase of under-dose. Furthermore, surgical removal of the implant is required if severe over-dosing occurs.

Emulsions are mixtures of two or more liquids that do not solubilize. Thus, the two or more liquids do not form a solution and an identifiable interface exists between the combined liquids. Emulsions may be macroemulsions, pseudo-emulsions, nanoemulsions or microemulsions. Emulsions may be used for parenteral delivery, ocular delivery, transdermal delivery, oral delivery, and the like.

FIG. 1A represents an example nanoemulsion droplet 100 having a single wall of phospholipids (monolayer) forming a hydrophilic exterior 120 and a hydrophobic interior 110. The monolayer wall of the nanoemulsion droplet 100 is formed from a single layer of phospholipids. The outer wall 120 is water-soluble due to the phosphate functionality while the interior 110 is fat-soluble due to the alkyl functionality. FIG. 1B represents multiple of the nanoemulsion droplets 100 in a continuous phase 150.

FIG. 2A represents a microemulsion droplet 200 having a single wall of phospholipids (monolayer) forming a hydrophilic exterior 220 and a hydrophobic interior 210. As with the nanoemulsion droplets 100, the monolayer wall of the microemulsion droplet 200 is formed from a single layer of phospholipids. In relation to the represented nanoemulsion droplets 100, the microemulsion droplets 200 are substantially smaller in diameter—which is often the case for microemulsions. In fact, the diameter of the microemulsion droplets 200 are reduced to where non-polar tails 230 of the monolayer phospholipids are “crushed” into each other, thus forming a more “solid” interior hydrophobic barrier than in the case of the nanoemulsion droplets 100 as represented in FIG. 1. FIG. 2B represents multiple microemulsion droplets 200 in a continuous phase 250. Also represented in the continuous phase 250 are a few individual phospholipid molecules 260 not incorporated into the microemulsion droplets 200.

Transdermal hormone creams are typically “pseudo-emulsions” with solid granules of the nonderivatized hormone not fully solubilized in the droplets of the emulsion forming the cream. In contrast to the larger droplet macro- and pseudo-emulsions, the smaller droplets of nanoemulsions and microemulsions provide the potential to provide better hormone delivery performance than conventionally available from macro- and pseudo-emulsions for either transdermal or oral adsorption; however, microemulsions are not readily made for nonderivatized hormones.

While the high-energy mixing, in the form of pressure (including shear forces), temperature, and combinations thereof, used to form nanoemulsions can provide the smaller droplets of a microemulsion, such nanoemulsions are not thermally stable, do not form shelf-stable microemulsions, and are like a macroemulsion in that the components of the nanoemulsion eventually separate into immiscible polar and non-polar liquids. Thus, as represented in FIG. 1 and FIG. 2, nanoemulsion droplets tend to be larger than microemulsion droplets as the nanoemulsion droplets continually expand in diameter after formation until the agglomerating droplets separate from the continuous phase.

Conventionally, macroemulsions, nanoemulsions, and microemulsions have been used for either oil-soluble or water-soluble deliverables, but have had limited success in solubilizing compounds having low solubility in oil and essentially no solubility in water. Deliverables, such as many nonderivatized hormones, have low solubility in oil and essentially no solubility in water, but often have good solubility in alcohol or in mixtures of alcohol and oil. However, if the nonderivatized hormone/alcohol or hormone/alcohol/oil mixture is dispersed along with surfactants into water-based solutions to form an emulsion, the alcohol tends to partition into the water and the nonderivatized hormone solubility enhancement provided by the alcohol, or the alcohol component of the alcohol/oil mixture is lost. This is believed attributable to the alcohol being extremely soluble in the water, in fact especially in relation to the oil if an alcohol/oil mixture is used.

Thus, the nonderivatized hormone loses significant bioavailability in such conventional emulsions, as once solubility in the alcohol or alcohol/oil mixture is lost, the nonderivatized hormone precipitates from the emulsion. In view of this disadvantage, conventionally, there has been little success in the development of oil-in-water (OIW) type microemulsions for nonderivatized hormone delivery, especially in the context of oral nonderivatized hormone delivery.

Unlike OIW emulsions (oil droplets in a water continuous phase), conventional water-in-oil emulsions (water droplets in an oil continuous phase—thus, an “invert emulsion”) have been made with nonderivatized hormones. One such example is found in U.S. Pat. Pub. 2009/0069279 (abandoned) to Astruc et al. Astruc describes using nonderivatized dehydroepiandrosterone (DHEA) in an invert emulsion using non-ingestible polar glycolic and hydroglycolic solvents dispersed with silicone-based emulsifiers into an oil medium. The reference recognizes the alcohol-soluble nature of nonderivatized DHEA and the difficulty of incorporating DHEA into an OIW emulsion. However, the WIO systems of Astruc cannot be made for human consumption because of the inedible constituents, thus being limited to dermal application.

Conventional emulsion delivery systems have traditionally addressed the inability to form true oil-in-water nonderivatized hormone emulsions by first derivatizing the hormone with ester functionality, thus substantially enhancing the oil-solubility of the hormone. The ester groups of the derivatized hormone provide increased oil-solubility to the hormone, thus permitting the esterified hormone to be dissolved in oils for injection or to be carried by conventional oil-in-water emulsion formulations.

A conventional example of hormone derivatization to increase oil solubility is the esterification of the steroidal hormone testosterone. Ester-derivatized testosterone is de-esterified to form bioavailable free testosterone after injection into a living mammal at different rates chiefly due to the release rate of the esterified hormone from the solubilizing excipient oil nodule formed at the injection site. While some variation in the release rate of the esterified hormone from the excipient oil may be attributable to injection technique and tissue variation, a significant factor determining the release rate of the esterified hormone after injection is the nature of the ester group attached to the testosterone.

For example, the propionate ester of testosterone is released from the injected excipient oil nodule much more rapidly than the cypionate ester. Because ester-derivatized testosterone is oil-soluble, in addition to injection with an oil excipient, ester-derivatized testosterone lends itself to conventional oil-in-water emulsion technologies that are used for oil-soluble deliverables. Disadvantages of such conventional methods may include, slowed and sporadic de-esterification of the oil-trapped hormone, stress placed on the liver by the required de-esterification process, the fact that not all hormones can be esterified in high yield, and the added complexity and hormone loss resulting from the esterification reaction.

An issue with conventional delivery systems, including nonderivatized hormone transdermal creams, nonderivatized hormone solid pellet implants, and derivatized hormone injectable oil preparations is that the release profile of the hormone into the bloodstream may not correlate well with the desired hormone dosing profile. Each of these conventional delivery systems is designed to eliminate the need to daily inject the nonderivatized hormone.

Injections including an excipient oil in combination with the derivatized hormone are designed to prevent having to daily inject the nonderivatized hormone by releasing the derivatized hormone from the oil excipient over time, thus permitting one or two injections per week to maintain a decaying, but somewhat level hormone concentration in the bloodstream. Solid pellet implants are designed to replace weekly or bi-weekly injections with quarterly surgical implants.

However, research indicates that such slowly decaying blood hormone concentrations over an extended period may not be desired. In fact, such injection of esterified testosterone dissolved in oil or implantation of constant release capsules may generate supraphysiological and/or constantly elevated testosterone concentrations in the blood that fail to provide the desired androgenic effects while increasing the likelihood of undesirable side effects.

For example, in “Testosterone in a cyclodextrin-containing formulation: behavioral and physiological effects of episode-like pulses in rats.” (Pharm Res. 1989 July; 6(7): 641-6) the authors demonstrated in castrated and intact rats that testosterone supplementation should mimic the natural episodic release by the testes to obtain the greatest improvement in androgen-sensitive behavior and physiology. Thus, testosterone supplementation should follow a “pulsed” regiment of multiple high doses that trail off rapidly throughout the day. The study also demonstrated that the testosterone effects were more pronounced when the high pulsed dosages were used periodically rather than when the same total amount of testosterone was equally divided among doses. The study also noted that both spermatogenesis and increased muscle weight were observed without substantial enlargement of the prostate.

In combination with other studies, the authors suggest that a testosterone dose that trails off in a slow and protracted manner over the course of a week due to a single injection with slowed release from the excipient oil nodule or the even longer trailing decay provided by the implantation of solid pellets may not be the proper path to optimal testosterone replacement therapy and may in fact be a contributor to the adverse effects presently associated with testosterone replacement therapy.

The dosing regimen used in the study required daily injections of hormone in an inclusion complex. While such dosing could be used for HRT, the required daily injections would be a deterrence to a large percentage of the population in need of HRT. While hormone creams allow for daily use without injection, the slow and variable uptake through the skin does not replicate the pulsed, rapid on-off blood hormone concentrations of the study. Furthermore, in the instance of derivatized hormones, the additional liver toxicity arising from de-esterification would be a further deterrence to using derivatized hormones in such a dosing regimen.

There is an ongoing need for simple and efficient materials and methods for oral delivery systems for delivering nonderivatized hormones having poor solubility in oil and essentially no solubility in water to the bloodstream. Conventional emulsion systems have traditionally had disadvantages including poor stability to cold and heat, particularly regarding maintaining the desired average droplet diameter in the emulsion, which is important for effective intra-oral delivery to the bloodstream, preventing phase separation of the oil and water components, and preventing dissociation of the deliverable from the emulsion. In addition to these disadvantages resulting in poor bioavailability of the deliverable, conventional emulsion systems also have the disadvantage of requiring too great a volume of the emulsion in relation to the mass or volume of the deliverable. These disadvantages have been especially true for the oral delivery of nonderivatized hormones to mammals, such as humans.

The microemulsions and methods of the present invention overcome at least one of the disadvantages associated with conventional delivery systems by allowing the convenient and reproducible oral delivery of nonderivatized, directly solubilized hormones to the bloodstream to achieve a desired dosing regimen, a significant and previously impractical even if pulsed androgenic activation is the desired result.

### SUMMARY

In one aspect, the invention provides a composition including an alcohol-soluble species, the alcohol-soluble species comprising dehydroepiandrosterone (DHEA), pregnenolone, and diindolylmethane (DIM); and a modified oil-in-water microemulsion including a modified oil phase and a modified polar continuous phase, where the alcohol-soluble species is solubilized in the modified oil phase, the modified oil phase comprising a phospholipid, a polyethylene glycol derivative, an oil, and an alcohol, and where the modified polar continuous phase comprises a sugar or sugar alcohol and water.

In another aspect of the invention, there is a method of making the composition of any one of the preceding claims, the method including combining the phospholipid, the polyethylene glycol derivative, the oil, and the alcohol to form an alcohol-lipid mixture; combining a sugar or sugar alcohol and water to form a modified polar continuous phase; and combining the alcohol-soluble species with the alcohol-lipid mixture and the modified polar continuous phase at atmospheric pressure to form the modified oil-in-water microemulsion.

In another aspect of the invention, there is a method of orally delivering alcohol-soluble species dehydroepiandrosterone and pregnenolone to the bloodstream of a human subject, the method comprising: introducing a composition comprising the alcohol-soluble species orally to a human subject, the composition comprising: alcohol-soluble species comprising dehydroepiandrosterone and pregnenolone; and a modified oil-in-water microemulsion comprising a modified oil phase and a modified polar continuous phase, where the alcohol-soluble species are solubilized in the modified oil phase, the modified oil phase comprising a phospholipid, a polyethylene glycol derivative, an oil, and an alcohol, and where the modified polar continuous phase comprises a sugar or sugar alcohol and water; and delivering the alcohol-soluble species dehydroepiandrosterone and pregnenolone to the bloodstream of the human subject, where within 60-minutes of the introducing the composition, approximately 2 mL of the composition provides the human subject a blood concentration increase from 200 to 500 ug/dL of the dehydroepiandrosterone or a metabolite of the dehydroepiandrosterone over a baseline pre-introducing bloodstream concentration.

In another aspect of the invention, there is a method of treating a female human subject in need of hormone replacement therapy with a pulsed dosage regimen, the method comprising: orally consuming daily for a treatment period of at least two weeks a modified oil-in-water microemulsion, the modified oil-in-water microemulsion comprising: alcohol-soluble species comprising an effective amount of dehydroepiandrosterone (DHEA) and of pregnenolone; and a modified oil-in-water microemulsion comprising a modified oil phase and a modified polar continuous phase, where the alcohol-soluble species are solubilized in the modified oil phase, the modified oil phase comprising a phospholipid, a polyethylene glycol derivative, an oil, and an alcohol, and where the modified polar continuous phase comprises a sugar or sugar alcohol and water; at least doubling a baseline dehydroepiandrosterone blood concentration in a bloodstream of the human subject within one hour of the orally consuming to produce an elevated dehydroepiandrosterone blood concentration; and reducing the elevated dehydroepiandrosterone blood concentration in the bloodstream of the human subject to the baseline dehydroepiandrosterone bloodstream concentration in the bloodstream of the human subject within three hours of the orally consuming.

Other compositions, methods, features, and advantages of the invention will be, or will become, apparent to one with skill in the art upon examination of the following figures and detailed description. It is intended that all such additional compositions, methods, features, and advantages be included within this description, be within the scope of the invention, and be protected by the claims that follow.

### BRIEF DESCRIPTION OF THE DRAWINGS

The invention can be better understood with reference to the following drawings and description. The components in the figures are not necessarily to scale and are not intended to accurately represent molecules or their interactions, emphasis instead being placed upon illustrating the principles of the invention.

FIG. 1A represents a nanoemulsion droplet having a single wall of phospholipids (monolayer) forming a hydrophilic exterior and a hydrophobic interior.

FIG. 1B represents multiple of the nanoemulsion droplets in a continuous phase.

FIG. 2A represents a microemulsion droplet having a single wall of phospholipids (monolayer) forming a hydrophilic exterior and a hydrophobic interior.

FIG. 2B represents multiple microemulsion droplets represented in a continuous phase.

FIG. 3 represents a method of making a MOIW microemulsion including an alcohol-soluble species.

FIG. 4 provides the results of a bioavailability duration analysis in graphical form for oral dosing of DHEA, an alcohol-soluble, nonderivatized hormone, adjusted to only show the increase in DHEA-S blood serum concentration over baseline DHEA-S blood serum concentration.

FIG. 5 provides the results of the comparative delivery efficiency of orally introduced, nonderivatized DHEA in graphical form adjusted to show the increase in DHEA-S blood serum concentration over baseline DHEA-S blood serum concentration.

FIG. 6 provides the results of a bioavailability uptake and duration analysis in graphical form for oral dosing of testosterone, an alcohol-soluble, nonderivatized hormone.

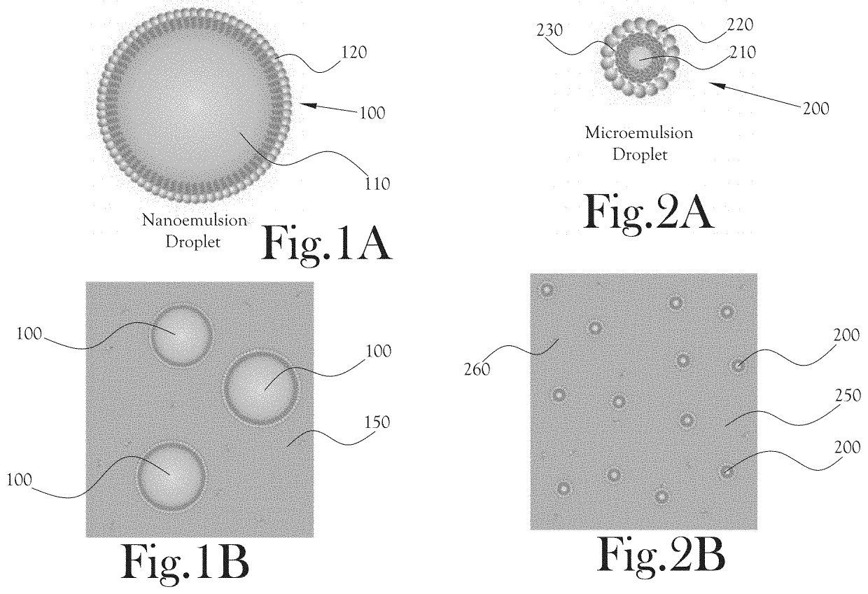

### DETAILED DESCRIPTION

Microemulsions are described where hydrophobic liquid droplets are distributed in a continuous hydrophilic liquid phase. In relation to conventional oil-in-water (OIW) microemulsions, the described microemulsions may be thought of as modified oil-in-water (MOIW) microemulsions, where both the “oil” and “water” phases of the microemulsion are modified. The oil phase droplets of the MOIW microemulsion are modified with alcohol and can solubilize alcohol-soluble species, including derivatized hormones. More preferably, the modified oil phase droplets of the MOIW microemulsion directly solubilize nonderivatized hormones. The polar continuous “water” phase of the MOIW microemulsion is modified with a sugar or sugar alcohol. Preferably, the modified polar continuous phase of the MOIW microemulsion is primarily a sugar or sugar alcohol phase. The modified oil phase droplets disperse into the modified polar continuous phase of the MOIW microemulsion.

The modified polar continuous phase is believed to allow the modified oil phase droplets of the microemulsion to incorporate and retain a high alcohol content. Thus, the modified polar continuous phase is believed to force the oil, alcohol, and alcohol-soluble species into the interior of the monolayer walls formed from a phospholipid and a polyethylene glycol derivative, thus into the hydrophobic core of the modified oil droplets, while the modified polar continuous phase including the sugar or sugar alcohol and water resides external to the monolayer.

Unlike the water continuous phase of a conventional OIW emulsion, the sugar or sugar alcohol of the modified polar continuous phase does not readily form an azeotrope with alcohol, and thus has a reduced ability to extract the alcohol from the oil droplets in relation to water. The hydrophobic portion of the monolayer wall formed from the tails of the phospholipid and in combination with the polyethylene glycol derivative in the described ratios also are believed to reduce alcohol loss from the oil droplets in relation to conventional OIW emulsions.

The retained high alcohol content of the modified oil phase droplets provided by the combination of the modified polar continuous phase with the hydrophobic monolayer is believed to increase the solubility of the alcohol-soluble species in the modified oil droplets of the MOIW microemulsion in relation to conventional OIW emulsions. This enhanced solubility of the alcohol-soluble species in the modified oil droplets of the MOIW is believed to reduce dissociation (e.g., recrystallization, precipitation, and like—thus separation) of the alcohol-soluble species from the oil droplets of the MOIW microemulsion during storage thus making the MOIW microemulsion a shelf-stable microemulsion that preferably is visually clear.

In the MOIW microemulsion, modified oil phase droplets including the alcohol-soluble species have an average droplet diameter of 1 to 100 nanometers and a preferable average droplet diameter of 5 to 50 nanometers. More preferably, the modified oil phase droplets of the MOIW microemulsion have an average droplet diameter from 7 to 30 nanometers.

The alcohol-soluble species of the MOIW microemulsions is a deliverable that may be delivered trans-mucosal (e.g., oral, intranasal, vaginal, or rectal) or transdermally via the MOIW microemulsion. In addition to directly solubilized nonderivatized hormones, derivatized hormones, such as esterified hormones, may be included in the microemulsion, in the event a greater hormone density in the microemulsion is desired.

The MOIW microemulsion can provide the uptake of the alcohol-soluble species to the bloodstream of a mammal through the oral and gastric mucosa, as well as transdermally through the skin. When the alcohol-soluble species is a nonderivatized hormone, such uptake to the bloodstream may be accomplished without the substantial modification and/or transformation of the nonderivatized hormone that has plagued prior, conventional OIW microemulsion attempts and without substantial stress on the liver.

Preferably, the MOIW microemulsion including the alcohol-soluble species is ingestible and edible. Thus, unlike suggested in the literature regarding WIO microemulsions, the described MOIW microemulsions unexpectedly provide therapeutically effective bloodstream concentrations of nonderivatized hormones, including testosterone, via oral delivery.

The ability of the MOIW microemulsion to deliver nonderivatized hormone alcohol-soluble species rapidly, efficiently, and without substantial modification and/or transformation provides for pulsed dosing regimens not practical with conventional delivery systems. For example, the benefits of a pulsed dosing regimen for testosterone are suggested from prior animal studies, where daily injected doses of testosterone were found to mimic the natural episodic release of testosterone from the testes and provided improvements in androgen-sensitive behavior and physiology. In contrast, introducing the same total amount of testosterone supplied by the multiple, daily injections as a single dose that is slowly released over an extended time resulted in unnatural, constantly elevated testosterone blood concentrations, which was suggested to be the cause of undesirable side effects associated with testosterone HRT.

A pulsed testosterone dosing regimen made possible by the MOIW microemulsion would include a daily, morning, intra-oral dose of testosterone. As testosterone is rapidly metabolized from the blood with about 90% metabolized within the first hour of introduction and the remainder metabolized within three hours, elevated blood testosterone levels would not exist but for a few hours each morning. The very short, elevated period in relation to the very long normal period should substantially reduce testicular atrophy and other undesirable side effects attributable to continuously elevated blood testosterone levels. While such a pulsed testosterone dosing regimen could be implemented through daily injection, such a regimen is made possible by the MOIW microemulsion without injection.

The MOIW microemulsion preferably includes a ratio of phospholipid, to oil, to polyethylene glycol derivative, to alcohol, to sugar or sugar alcohol, and to water of 1:2:0.6-3.3:4:10.5:1-1.6 by weight, with deviations up to 20% by weight being included, and with deviations up to 10% by weight being more preferred, thus 1:2:0.6-3.3:4:10.5:1-1.6±20% by weight or 1:2:0.6-3.3:4:10.5:1-1.6±10% preferred by weight.

The alcohol-soluble species is preferably included in the MOIW microemulsion at a ratio of oil to alcohol-soluble species of 1:0.02 to 0.5 by weight, with a ratio of oil to alcohol-soluble species of 1:0.1 to 0.3 by weight being preferred with deviations up to 10% by weight being included, and with deviations up to 5% by weight being more preferred, thus 1:0.02 to 0.3±10% by weight or 1:0.02 to 0.3±5% preferred by weight.

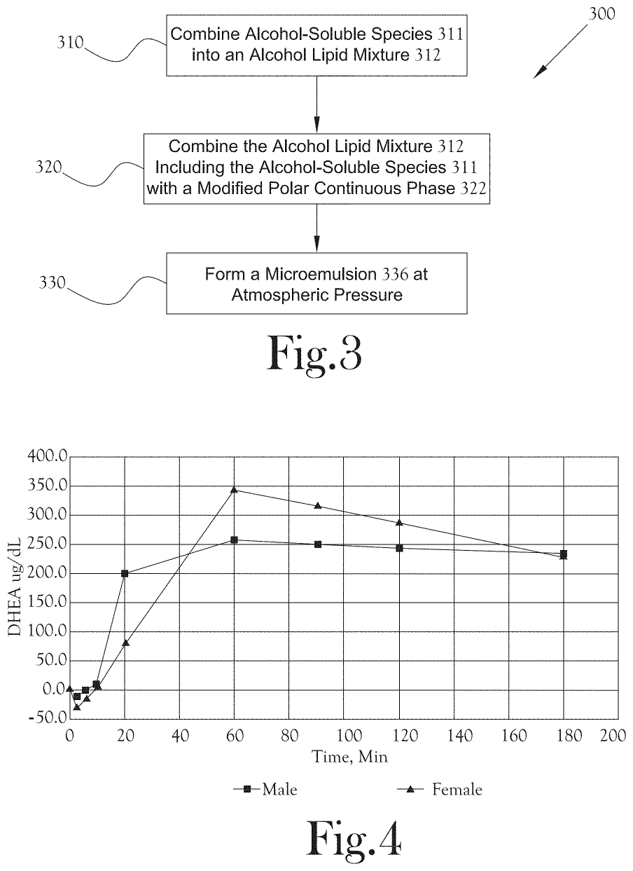

FIG. 3 represents a method 300 of making a MOIW microemulsion 336 including an alcohol-soluble species 311. In 310, the alcohol-soluble species 311 is combined into an alcohol-lipid mixture 312 including a polyethylene glycol derivative, a phospholipid, an oil, and an alcohol. In 320, the alcohol-lipid mixture 312 including the alcohol-soluble species 311 is combined with a modified polar continuous phase 322 including the sugar or sugar alcohol and water. The alcohol-lipid mixture 312 including the alcohol-soluble species 311 may be considered a modified oil phase dispersed in the modified polar continuous phase 322, which may be thought of as a modified water phase.

In 330, the microemulsion 336 including the alcohol-soluble species 311 is formed by mixing at atmospheric pressure. Unlike in nanoemulsions, the microemulsion 336 may be formed at atmospheric pressure without needing the energy of elevated pressures and/or shear forces to form. Although the microemulsion 336 could be formed using elevated pressure and/or shear forces as used in forming nanoemulsions, the result eventually will be the microemulsion 336, as unlike in a nanoemulsion that begins the dissociation process after formation—even if dissociation is very slow, the microemulsion 336 is thermally stable at room temperature and pressure after formation. Thus, formation of the microemulsion 336 dispenses with the undesirable use of elevated pressures and/or shear forces during formation, and is shelf-stable after formation.

While the method 300 represents the alcohol-soluble species 311 first being combined with the alcohol-lipid mixture 312, the alcohol-lipid mixture 312 and the polar continuous phase 322 may first be combined and the alcohol-soluble species 311 then added to form the microemulsion 336 (not shown). This step rearrangement is possible as the modified oil and modified polar continuous phases will “self-assemble” droplets including the alcohol-soluble species to form the microemulsion 336 at atmospheric pressure.

In addition to the alcohol-soluble species 311, the microemulsion 336 may include additional deliverables that are soluble in water or oil. However, the microemulsion 336 has the unexpected ability to orally deliver therapeutically effective concentrations of the alcohol-soluble species to the bloodstream of a living mammal.

The alcohol-soluble species 311 includes nonderivatized hormones, polyphenols, plant sterols, and amines. The alcohol-soluble species is solubilized in the droplets of the microemulsion 336, thus in the alcohol-lipid mixture 312. Preferably, the alcohol-soluble species 311 constitutes from 0.2% to 5% of the microemulsion 336 by weight. However, to provide a visually clear emulsion with the widest range of alcohol-soluble species, weight percentages of the alcohol-soluble species 311 from 0.2% to 3% are preferred, with weight percentages from 0.25% to 3% being more preferred. For nonderivatized hormones, weight percent in the microemulsion 336 from 0.2% to 1.8% are readily achieved, with a weight percent range from 0.25% to 1.5% being readily achieved for nonderivatized testosterone.

Preferable alcohol-soluble nonderivatized hormones include testosterone, dehydroepiandrosterone (3-beta-hydroxyandrosteron-5-en-17-one) (DHEA), dihydrotestosterone (DHT), 7-keto DHEA, pregnenolone, androstenedione (AD), androstenediol, progesterone, estradiol, estrone, estriol, and cortisol. More preferred nonderivatized hormones are testosterone and DHEA. At present, the most preferred nonderivatized hormone is testosterone. Preferable alcohol-soluble polyphenols include chrysin, hesperetin, and apigenin. Preferable alcohol-soluble plant sterols include Tribulus terrestris and yohimbe, while preferable alcohol-soluble amines include diindolylmethane (DIM).

The alcohol lipid mixture 312 may include an oil-soluble deliverable specie or species that are more soluble in oil than the alcohol-soluble species 311. Such oil-soluble deliverables are solubilized in the modified oil phase droplets of the microemulsion, thus in the alcohol lipid mixture 312 with the alcohol-soluble species 311.

Oil-soluble deliverable species include derivatized hormones, cannabis extracts, and terpenes. Preferable derivatized hormones include testosterone-propionate, testosterone-cypionate, testosterone-enanthate, and testosterone-phenylpropionate. More preferred derivatized hormones are testosterone-propionate and testosterone-cypionate. At present, the most preferred derivatized hormone is testosterone-cypionate. Preferable cannabis extracts include cannabidiol (CBD), tetrahydrocannabinol (THC), and other cannabinoids including cannabinol (CBN), cannabigerol (CBG), tetrahydrocannabivarin (THCV), cannabidivarin (CBDV), and cannabichromene (CBC).

Preferable terpenes include monoterpenes (incorporate two isoprene units and have the molecular formula C₁₀H₁₆), monoterpenoids, diterpenes (incorporate four isoprene units and often have the molecular formula C₂₀H₃₂), and diterpenoids. Preferable terpenes include limonene, pinene, linalool, beta-caryophyllene, retinol, phytol, myrcene, humulene, ocimene, terpinolene, geraniol, and geranylgeraniol.

The modified polar continuous phase 322 may include a water-soluble deliverable specie or species that are more soluble in water than the alcohol-soluble species 311. Such water-soluble deliverables are solubilized in the modified polar continuous phase 322 of the microemulsion 336. Thus, in the carrier liquid of the microemulsion 336.

The phospholipid and the polyethylene glycol derivative in combination form the boundary between the modified polar continuous phase and the interior of the modified oil phase droplets of the microemulsion 336. To maintain the desired alcohol concentration within the droplets, thus reducing the likelihood of losing the alcohol to the modified polar continuous phase and the associated dissociation of the alcohol-soluble species from the droplets, the phospholipid, polyethylene glycol derivative, and the ratio between the two are important, as previously discussed.

The phospholipid of the alcohol-lipid mixture 312 is a glycerophospholipid preferably isolated from lecithin. As the phospholipid is preferably a lecithin isolate, the named isolates preferably include 80% (w/w) of the specified phospholipid with the remaining constituents being one or more additional phospholipids isolated from the lecithin or other lecithin isolates. Preferred phospholipid lecithin isolates include phosphatidylcholine (PC), phosphatidylethanolamine (PE), phosphatidylinositol (PI), ceramide phosphoryl ethanolamine (Cer-PE), ceramide phosphoryl choline (SPH), and combinations thereof, with PC, PE, and combinations thereof being more preferred. However, all phospholipid lecithin isolates are unexpectedly not interchangeable in forming visually clear, shelf-stable MOIW microemulsions, as the phosphatidylserine (PS) and phosphatic acid (PA) isolates are not useful when both visually clear and shelf-stable MOIW microemulsions are desired. When the alcohol-soluble species 311 is nonderivatized testosterone, the phospholipid is preferably PC.

The phospholipid may be present in the microemulsion 336 from 3% to 10% on a weight basis. Preferably, the phospholipid constitutes from 4% to 8% of the microemulsion 336 on a weight basis. When the alcohol-soluble species is nonderivatized testosterone, the phospholipid constitutes from 4% to 6% of the microemulsion 336 on a weight basis.

The polyethylene glycol derivative of the alcohol-lipid mixture 312 may be a polyethylene glycol modified vitamin E, such as tocopheryl polyethylene glycol succinate 1000 (TPGS), polysorbate 40, polysorbate 60, or polysorbate 80. Preferably, the polyethylene glycol derivate is TPGS, polysorbate 60, or polysorbate 80. More preferably, the polyethylene glycol derivative is TPGS or polysorbate 80. When the alcohol-soluble species is nonderivatized testosterone, the preferred polyethylene glycol derivative is TPGS.

The polyethylene glycol derivative may be present in the microemulsion 336 from 5% to 14% on a weight basis. Preferably, the polyethylene glycol derivative constitutes from 6% to 12% of the microemulsion 336 on a weight basis. When the alcohol-soluble species is nonderivatized testosterone, the polyethylene glycol derivative constitutes from 9% to 11% of the microemulsion 336 on a weight basis.

TPGS, polysorbate 20, polysorbate 40, polysorbate 60, and polysorbate 80 are often thought of as interchangeable surfactants. This was determined not to be the case in the formation of the described microemulsion 336 when a visually clear, shelf-stable microemulsion is desired.

When used in conjunction with the phospholipid, TPGS resulted in visually clear, shelf-stable microemulsions at phospholipid to TPGS ratios of approximately 1:0.4 to 1:4 by weight, with preferred shelf-stable MOIW microemulsions being formed at ratios of 1:1.6 to 1:4 by weight. When used in conjunction with the phospholipid, polysorbate 20 did not form visually clear, shelf-stable microemulsions. When used in combination with the phospholipid, polysorbate 40 resulted in visually clear, shelf-stable microemulsions at PC to polysorbate 40 ratios of approximately 1:2 to 1:3 by weight, with preferred shelf-stable MOIW microemulsions being formed at a ratio of approximately 1:3 by weight. When used in combination with the phospholipid, polysorbate 60 resulted in visually clear, shelf-stable microemulsions at phospholipid to polysorbate 60 ratios of approximately 1:2 to 1:4 by weight, with preferred shelf-stable MOIW microemulsions being formed at a ratio of 1:2 to 1:3 by weight. When used in combination with the phospholipid, polysorbate 80 resulted in visually clear, shelf-stable microemulsions at phospholipid to polysorbate 80 ratios of approximately 1:0.4 to 1:4 by weight, with preferred shelf-stable MOIW microemulsions being formed at a ratio of 1:0.6 to 1:4 by weight.

These results establish that the multiple polyethylene glycol derivatives are unexpectedly not interchangeable in forming visually clear, shelf-stable MOIW microemulsions. In fact, polysorbate 20 is not useful. Furthermore, TPGS and polysorbate 80 are the preferred polyethylene glycol derivatives as in combination with the phospholipid, they provide the desired visually clear, shelf-stable microemulsions over the widest alcohol-soluble species concentration range.

The alcohol-lipid mixture 312 preferably includes at least one oil held within the phospholipid/polyethylene glycol derivative monolayer. The oil may be an MCT oil, a citrus oil, and combinations thereof. MCT oils are triglycerides whose fatty acids have an aliphatic tail of 6-12 carbon atoms. Preferable MCT oils include caproic acid (hexanoic acid), caprylic acid (octanoic acid), capric acid (decanoic acid), lauric acid (dodecanoic acid), and combinations thereof. More preferred MCT oils include caprylic acid, capric acid, and combinations thereof. Preferred citrus oils include orange oil, lemon oil, and combinations thereof. When the alcohol-soluble species is nonderivatized testosterone, the oil is preferably a combination of caprylic and capric acids.

The oil may be present in the microemulsion 336 from 5% to 15% on a weight basis. Preferably, the oil constitutes from 7% to 13% of the microemulsion 336 on a weight basis. When the alcohol-soluble species is nonderivatized testosterone, the oil constitutes from 9% to 11% of the microemulsion 336 on a weight basis.

The microemulsion 336 includes at least one alcohol. The preferable alcohol is food grade as the microemulsion 336 is preferably edible. Preferably, the alcohol is ethanol, with USP food grade 190 proof (95% ethanol, 5% water by weight) ethanol being more preferred. Alcohol water contents greater than 10% are less preferred, as then the additional water should be considered in relation to the total water content of the microemulsion 336 to prevent dissociation of the alcohol-soluble species from the modified oil phase droplets as discussed further below.

The alcohol may be present in the microemulsion 336 from 5% to 25% on a weight basis. Preferably, the alcohol constitutes from 10% to 23% of the microemulsion 336 on a weight basis. When the alcohol-soluble species is nonderivatized testosterone, the alcohol constitutes from 17% to 22% of the microemulsion 336 on a weight basis.

The modified oil phase droplets of the microemulsion 336 may be considered to have a high alcohol content, thus having an oil to alcohol weight ratio of from 1:1.5 to 1:4, preferably from 1:1.5 to 1:3 by weight.

The modified polar continuous phase 322 includes a sugar or sugar alcohol and water. By “sugar or sugar alcohol” it is meant a sugar or a sugar alcohol preferably including from 3 to 12 carbon atoms that is a liquid at room temperature and pressure or soluble in water at room temperature and pressure. Preferable sugars include sucrose, cane sugar, and pure maple syrup, with pure maple syrup being preferred due to the inclusion of tree resins. Preferable sugar alcohols have from 3 to 6 carbon atoms and include glycerol (glycerin).

While one could expect additional sugar alcohols, including xylitol, erythritol, mannitol, and sorbitol to be useful in forming the microemulsion 336, all sugar alcohols are unexpectedly not interchangeable in forming visually clear, shelf-stable MOIW microemulsions, as xylitol, erythritol, mannitol, and sorbitol are not useful when both visually clear and shelf-stable microemulsions are desired. Thus, preferred sugar or sugar alcohols include sucrose, cane sugar, pure maple syrup, glycerol, and combinations thereof. More preferred sugar or sugar alcohols include pure maple syrup, glycerol, and combinations thereof. Presently, the most preferred sugar or sugar alcohol is glycerol.

When the sugar or sugar alcohol is glycerol, the ratio of glycerol to water is from 12:1 to 8:1 by weight, preferably 10:1 by weight with deviations up to 20% by weight being included, and with deviations up to 10% by weight being more preferred, thus 10:1±20% by weight or 10:1±10% preferred by weight. When the sugar or sugar alcohol is pure maple syrup, sucrose, or cane sugar, and water is present in the syrup or used to solubilize the sucrose or cane sugar, this additional water becomes part of the water constituent of the microemulsion 336 and is thus included in the sugar or sugar alcohol to water weight ratio as water.

When the sugar or sugar alcohol is glycerol, the glycerol may be present in the microemulsion 336 from 43% to 56% on a weight basis with a total water content of 5% to 10% by weight. Preferably, the glycerol constitutes from 45% to 52% of the microemulsion 336 on a weight basis with a total water content of 5% to 10% by weight. When the alcohol-soluble species is nonderivatized testosterone, the glycerol constitutes from 48% to 52% of the microemulsion 336 on a weight basis.

The water of the polar continuous phase 332 is present in the microemulsion 336 from 2% to 10% on a weight basis. Preferably, water is present from 4% to 10% on a weight basis in the microemulsions 336. More preferably, water may be present in the microemulsion 336 from 4% to 8% on a weight basis. When the alcohol-soluble species is nonderivatized testosterone, water is present in the microemulsion 336 from 4% to 6% on a weight basis. Water contents greater than 12% and in some instances in excess of 10% up to the 12% limit in the microemulsion 336 on a weight basis may result in dissociation of the alcohol-soluble species from the droplets, and thus non-shelf-stable MOIW microemulsions resulting from an excessive loss of the alcohol from the droplets.

While not shown in FIG. 3, the oil may be reduced to the point of omission from the method 300 if the amount of the sugar or sugar alcohol is simultaneously increased. For example, if the microemulsion 336 is formed with 5% oil by weight and 56% sugar alcohol by weight, a MOIW microemulsion could be formed with 3% oil by weight and 58% sugar or sugar alcohol by weight or with 0% oil and up to 63% sugar or sugar alcohol by weight. When a MOIW microemulsion includes less than 5% oil, 53% to 63% sugar or sugar alcohol by weight is preferred. When a MOIW microemulsion includes 0% oil, 57% to 63% sugar or sugar alcohol by weight is preferred. While these “reduced oil” microemulsions will be visually clear and shelf-stable, the average droplet diameters will be on the upper end of the scale, thus closer to 100 nanometers, and thus will be less effective at oral delivery of the deliverable. Such a “reduced oil” MOIW microemulsion preferably has a ratio of phospholipid, to polyethylene glycol derivative, to alcohol, to sugar or sugar alcohol, and to water of 1:0.6-3.3:4:10.5:1-1.6 by weight, with deviations up to 20% by weight being included, and with deviations up to 10% by weight being more preferred, thus 1:0.6-3.3:4:10.5:1-1.6±20% by weight or 1:0.6-3.3:4:10.5:1-1.6±10% preferred by weight.

The microemulsion 336 may optionally include other ingredients or “adjuvants” that are chemically compatible with the alcohol-soluble species and do not substantially interfere with the separation between the modified oil and water phases of the microemulsion. Such adjuvants may include hydrophilic or lipophilic gelling agents, thickeners, preservatives, antioxidants, electrolytes, perfumes, fillers, and pigments. Other adjuvants may be used in the microemulsion.

The following examples are provided to illustrate one or more preferred embodiments of the invention. Numerous variations can be made to the following examples that lie within the scope of the invention.

### EXAMPLES

### Example 1: Constituents of a MOIW Microemulsion Including the Nonderivatized Hormone DHEA

A MOIW microemulsion was prepared having a 1 mL total volume. The MOIW microemulsion included approximately 10 mg of the nonderivatized hormone DHEA. The MOIW microemulsion also included from 30 mg to 100 mg of PC, from 150 mg to 250 mg of ethanol, from 400 mg to 650 mg of glycerin, and from 50 mg to 150 mg of medium chain triglycerides. TPGS was included to provide the desired physical structures in the MOIW microemulsion. In addition to these ingredients, the MOIW microemulsion included enough water to provide a total emulsion volume of 1 mL.

### Example 2: A Method of Making a MOIW Microemulsion Including the Nonderivatized Hormone DHEA

Approximately 10 mg of nonderivatized DHEA was combined in MCT oil and then combined with TPGS, PC, glycerin, and ethanol in water. The combination was then mixed to form a MOIW microemulsion including the nonderivatized hormone DHEA having a total volume of 1 mL.

### Example 3: Bioavailability Uptake and Duration for Intra-Oral Delivery of Nonderivatized DHEA

Nonderivatized DHEA, an alcohol-soluble hormone, was incorporated into a MOIW microemulsion in accord with Example 2. On an empty stomach, adult male and female human subjects placed 2 mL of the MOIW microemulsion including the nonderivatized DHEA under the tongue. The subjects held the MOIW microemulsion under the tongue for approximately 30 seconds to 2 minutes before swallowing. Blood samples were collected before the MOIW microemulsion was administered and at varying time intervals between approximately 20 and 180 minutes after administration of the MOIW microemulsion. The collected blood samples were analyzed for the blood serum concentration of DHEA-S, the sulfated congener of DHEA which is an initial product produced by the body from metabolizing DHEA.

FIG. 4 provides the results of the bioavailability uptake and duration analysis in graphical form for oral intra-oral dosing of DHEA adjusted to only show the increase in DHEA-S blood serum concentration over baseline DHEA-S blood serum concentration.

For both the male and female subjects, the MOIW microemulsion increased the blood serum DHEA-S concentration to a maximum approximately 60-minutes post introduction and maintained a near level blood serum concentration out to the 180-minute study end time. Baseline DHEA-S concentrations were at approximately 200 to 250 microgram (ug) per milliliter (mL) of blood, thus the increase in DHEA-S from approximately 200 to 300 ug/dL over the timeframe of the study was significant.

### Example 4: Comparative Delivery Efficiency of Orally Introduced Nonderivatized DHEA

The approximate percentages of DHEA delivered to the blood by the MOIW microemulsion was compared to DHEA orally delivered in crystalline and micronized forms. The comparative data used for the conventional crystalline and micronized formulations of DHEA was taken from “Delivery of dehydroepiandrosterone to premenopausal women: Effects of micronization and nonoral administration,” Casson, et al., Am J Obstet Gynecol, February 1996, Vol. 174, No. 2, pp. 649-653.

Regarding the conventional data used for comparison, the authors report in Casson that the micronized DHEA was prepared by micronizing pharmacopoeia-grade DHEA obtained from Sigma Chemical Company, St. Louis and compounding into DHEA tablets containing 300 mg per dose in a wax-vegetable oil matrix, with a silica-based excipient. (Casson, pg. 650). Identical tablets containing crystalline tablets also were prepared. Id. The tablets were administered to females in the midfollicular phase of the menstrual cycle after 8 hours of fasting. Id. Thus, in relation to the MOIW microemulsion which included 20 mg of DHEA per intra-oral dose, each tablet form dose from Caisson included either 300 mg or 150 mg of DHEA.

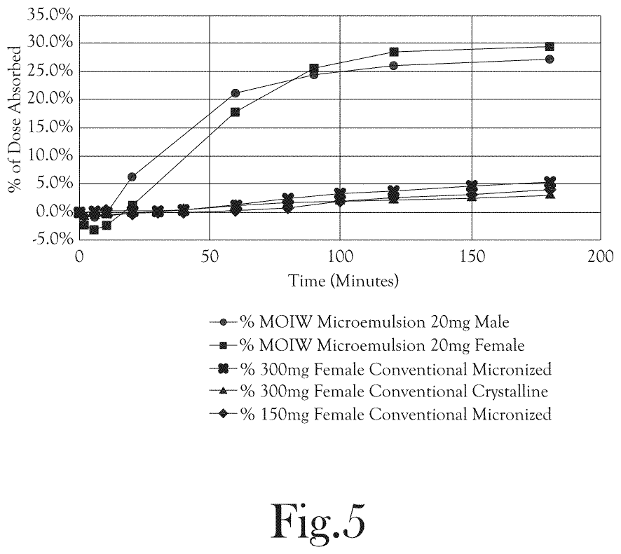

FIG. 5 provides the results of the comparative delivery efficiency of orally introduced, nonderivatized DHEA in graphical form adjusted to show the increase in DHEA-S blood serum concentration over baseline DHEA-S blood serum concentration. To facilitate the comparison, the presumption was made that each human subject had a blood volume of approximately 4.7 Liters including about 55% serum (plasma without clotting factors) by volume. Being applied across both the present MOIW microemulsion and conventional publication data, this presumption of human subject blood volume is not believed to alter the relationship of the underlying comparison.

While the tablets of Casson generally provided higher DHEA-S blood concentrations due to the substantially higher dosage amount than used in the MOIW microemulsion, a substantial difference in delivery efficiency (dose amount in relation to amount delivered to bloodstream) was observed when the 300 mg and 150 mg Casson DHEA dosages were considered in relation to the 20 mg MOIW microemulsion DHEA dosage. Delivery efficiency is important not only from the perspective of potentially reducing stress on the liver, but in relation to manufacturing cost.

As shown in FIG. 5, of the 300 mg of crystalline DHEA orally delivered in Casson, approximately 1% by weight was delivered to the blood approximately 60-minutes post introduction, with approximately 3% by weight being delivered within 180 minutes. For the micronized DHEA 300 mg and 150 mg doses of Casson, approximately 0.4-1% by weight was delivered to the blood approximately 60-minutes post introduction, with approximately 4-5% by weight being delivered within 180 minutes.

The difference in delivery efficiency with the 20 mg DHEA MOIW microemulsion dose was significant, where approximately 17-21% (male-female) by weight was delivered to the blood approximately 60-minutes post introduction, with 28-30% (male-female) by weight being delivered within 180-minutes. Thus, the MOIW microemulsion was able to deliver at least 14% by weight of the dosage amount within approximately 60-minutes, preferably at least 16% by weight of the dosage amount. The MOIW microemulsion also was able to deliver at least 25% by weight of the dosage amount within approximately 180-minutes, preferably at least 27% by weight of the dosage amount.

The improvement provided by the MOIW microemulsion for oral delivery in relation to the conventional dosage forms was substantial. At approximately 60-minutes post introduction, the MOIW microemulsion provided an approximately 17 times increase in delivery efficiency over the conventional dosage forms. By approximately 180-minutes, the MOIW microemulsion provided an approximately 5 times increase in delivery efficiency over the conventional dosage forms. While the rate of bloodstream delivery increased for the conventional dosage forms between the 60- and 180-minute times, the conventional forms failed to approach the “area under the curve” or total delivery of the MOIW microemulsion, which was greater than 5 times as much.

### Example 5: Bioavailability Uptake and Duration for Intra-Oral Delivery of Nonderivatized Testosterone

Nonderivatized Testosterone, an alcohol-soluble hormone, was incorporated into a MOIW microemulsion similarly to DHEA in accord with Example 2 except that approximately 12.5 mg of nonderivatized testosterone was used in 1 mL of the MOIW microemulsion. On an empty stomach, an adult male human subject placed 1 mL of the MOIW microemulsion including the nonderivatized testosterone under the tongue. The subjects held the MOIW microemulsion under the tongue for approximately 90 seconds before swallowing. Blood samples were collected before the MOIW microemulsion was administered and at varying time intervals between approximately 15 and 180 minutes after administration of the MOIW microemulsion. The collected blood samples were analyzed for the blood serum concentration of total testosterone.

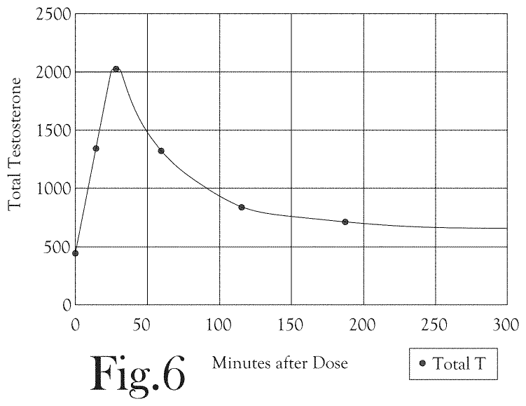

FIG. 6 provides the results of the bioavailability uptake and duration analysis in graphical form for oral intra-oral dosing of testosterone. For the male subject, the MOIW microemulsion increased the blood serum testosterone concentration to a maximum approximately 30-minutes post introduction. Baseline testosterone concentration was at approximately 500 nanograms (ng) per deciliter (dL) of blood, thus the increase in testosterone from approximately 500 to 1000 ng/dL was significant.

While the preceding uptake and duration examples are in the context DHEA and testosterone, we believe that the uptake performance for other nonderivatized hormone alcohol-soluble species would be similar in combination with the MOIW microemulsion. While the preceding delivery efficiency example is in the context of DHEA, we believe that similar delivery efficiency would be attained with other nonderivatized hormone alcohol-soluble species in combination with the MOIW microemulsion. However, the experimental delivery efficiency data for a nonderivatized hormone alcohol-soluble species such as testosterone would look different than recorded for DHEA as testosterone is metabolized from the blood much more rapidly than DHEA. Similarly, the experimental delivery efficiency data for a nonderivatized hormone alcohol-soluble species such as progesterone would be expected to approximate that recorded for DHEA as progesterone is metabolized from the blood at a rate similar to DHEA.

### Prophetic Example 6: Pulsed Dosing for Intra-Oral Delivery of Nonderivatized Testosterone

A human male subject in need of testosterone HRT intra-orally consumes 1 mL of the MOIW microemulsion including approximately 12.5 mg of nonderivatized testosterone daily. Blood testosterone concentrations reach an approximate concentration of 1500-2000 ng/dL within the first hour of consumption and decay to a baseline testosterone concentration within three hours of consumption. The daily MOIW microemulsion testosterone pulsed dosing regimen significantly reduces testicular atrophy in the male subject in relation to that normally observed with conventional HRT therapy, but provides the desired androgenic effects.

To provide a clear and more consistent understanding of the specification and claims of this application, the following definitions are provided.

Intra-oral delivery means that a substantial portion of the delivery into the bloodstream that occurs upon oral administration of the liquid including the deliverable occurs by transmucosal absorption through the mouth, throat and esophagus before the liquid reaches the stomach. For droplets to be considered suitable for intra-oral delivery, the average droplet diameter is at most 125 nm. Intra-oral delivery is believed to increase with decreasing average droplet diameter, with average droplet diameters of approximately 25 nm being preferred.

An alcohol-soluble species is a species that is insoluble in water and has a greater solubility in ethanol than in medium chain triglyceride (MCT) oils. For example, the nonderivatized hormone DHEA is soluble in ethanol up to approximately 150 mg/mL, thus being freely soluble, while having a solubility in MCT oil of only up to approximately 10 mg/mL, thus being only sparingly soluble. Alcohol-soluble species are preferably pharmacologically active, more preferably are a drug or a supplement, and neither include nor are water. Thus, liquids and solids may exist that technically are soluble in alcohol, but because they also are soluble in water or more or equivalently soluble in MCT oils than in ethanol are not “alcohol-soluble species”.

Nonderivatized hormones are chemically identical to hormones made by the human body and are not synthetically modified with fatty esters or other pendant groups.

Directly solubilize the nonderivatized hormone means that unlike in conventional systems, the nonderivatized hormone does not require synthetic conversion to an esterified state to be solubilized, thus the microemulsion “directly solubilizes” the nonderivatized hormone.

Phosphatidylcholine (PC) molecules are a subset of the larger set of phospholipids and are commonly used to form liposomes in water. When placed in water without other constituents, PC forms liposomes. The application of sufficient shear forces to the PC liposomes can produce monolayer structures, including micelles. PC has a head that is water-soluble and a tail that is much less water-soluble in relation to the head. PC is a neutral lipid, but carries an electric dipole moment of about 10 D between the head and the tail, making the molecule itself polar.

Tocopheryl polyethylene glycol succinate 1000 (TPGS) is generally considered a surfactant having a non-polar, oil-soluble “Vitamin E” tail and a polar, water-soluble polyethylene glycol head. TPGS is a member of the polyethylene glycol derivatives that also include polysorbate 20, 40, 60, and 80.

Room temperature and pressure means from 20 to 27 degrees Celsius at approximately 100 kPa.

Solid means a substance that is not a liquid or a gas at room temperature and pressure. A solid substance may have one of a variety of forms, including a monolithic solid, a powder, a gel, or a paste.

Liquid means as substance that is not a solid or a gas at room temperature and pressure. A liquid is an incompressible substance that flows to take on the shape of its container.

Solutions lack an identifiable interface between the solubilized molecules and the solvent. In solutions, the solubilized molecules are in direct contact with the solvent.

Solubilized means that the alcohol-soluble species to be delivered is in the solution of the droplet. When solubilized, dissociation (thus, liquid separation or solid formation) of the alcohol-soluble species does not result in droplet average particle diameters greater than 200 nm as determined by DLS and discussed further below, or by the formation of precipitated crystals of the alcohol-soluble species visible with the naked eye. Thus, if either average particle diameters greater than 200 nm or precipitated crystals visible to the naked eye form, the alcohol-soluble species is not solubilized in the solution of the droplet. If an alcohol-soluble species is not solubilized in the solution, it is insoluble in the solution. In many respects, solubility may be thought of as a concentration dependent continuum. For example, the following descriptive terms may be used to express solubility of a solute in a solvent (grams solid/mL of solvent) at 25 degrees Celsius:

TABLE 1

| Descriptive Level | Parts solvent per 1 part of solute |
|---|---|
| Very Soluble | Less than 1 |
| Freely Soluble | From 1 to 10 |
| Soluble | From 10 to 30 |
| Sparingly Soluble | From 30 to 100 |
| Slightly Soluble | From 100 to 1000 |
| Very Slightly Soluble | From 1000 to 10,000 |
| Insoluble | More than 10,000 |

Dissociation occurs when a previously solubilized solid or liquid leaves a solution and is no longer in direct contact with a solvent of the solution. Dissociation of solids from the solvent occurs through recrystallization, precipitation, and the like. Dissociation of liquids from the solvent occurs through separation and the formation of a visible meniscus between the solvent and the dissociated liquid.

A shelf-stable microemulsion may be determined in one of two ways. One way to establish that a microemulsion stored in a sealed container substantially excluding air and moisture is shelf-stable is when dissociation of a solid does not occur and the oil phase droplets in the water do not change in average diameter by more than +/−20% at about 25° C. for a time period of at least 3 months to 2 years, preferably for a time period of at least 6 months to 2 years, and more preferably, for a time period of at least 1 year to 2 years. Another way to establish that a microemulsion is shelf-stable is when dissociation of a solid does not occur and the oil phase droplets in the water do not separate into a visibly distinct phase with a visible meniscus when stored in a sealed container substantially excluding air and moisture at about 25° C. for a time period of at least 6 months to 2 years, and more preferably, for a time period of at least 1 year to 2 years. Either type of dissociation means that the microemulsion is not shelf-stable.

A visually clear microemulsion has an average particle diameter of 200 nm and less and lacks precipitated solid crystals visible to the naked eye.

Emulsions are mixtures of two or more liquids that do not solubilize. Thus, one of the liquids carries droplets of the second liquid. The droplets of the second liquid may be said to be dispersed in a continuous phase of the first liquid. An interface, separation, or boundary layer exists between the carrier liquid (continuous phase) and the droplets of the second liquid. Emulsions may be macroemulsions, pseudo-emulsions, microemulsions, or nanoemulsions. The primary difference between the emulsion types is the size (average diameter) of the droplets dispersed in the continuous phase and whether the droplets are thermodynamically stable in the continuous phase. Macroemulsions and pseudo-emulsions have average droplet diameters from 1 to 20 micrometers. Microemulsions and nanoemulsions have smaller average droplet diameters in the continuous phase than macroemulsions and pseudo-emulsions. Microemulsions are thermodynamically stable while nanoemulsions are not even though their average droplet diameters may overlap in size.

Droplets or liquid particles are formed by the hydrophobic “oil” phase of a microemulsion and are carried by the hydrophilic continuous phase. The exterior of the droplets is defined by a boundary layer that surrounds the volume of each liquid droplet. The boundary layer of a droplet defines the exterior surface of the droplets forming the dispersed oil phase of the microemulsion. The continuous phase of the microemulsion resides exterior to the boundary layer of the droplets, and thus, carries the droplets.

Macroemulsions are thermodynamically unstable but kinetically stable dispersions of oil in water, with oil being defined as any water-insoluble liquid. By thermodynamically unstable it is meant that once created, the macroemulsion is always reverting to the original, immiscible state of the oil and water constituents (demulsification), but this break down is slow enough (thus, kinetically “stable”) that the macroemulsion may be considered stable from an intended use practicality perspective. Macroemulsions scatter light effectively and therefore appear milky, because their droplets are greater in diameter than the wavelength of visible light. The IUPAC definition of a macroemulsion is an “emulsion in which the particles of the dispersed phase have diameters from approximately 1 to 100 micrometers. Macroemulsions comprise large droplets and thus are “unstable” in the sense that the droplets sediment or float, depending on the densities of the dispersed phase and dispersion medium.”

Pseudo-emulsions are dispersions of oil in water, with oil being defined as any water-insoluble liquid, including tiny (micronized) solid granules that are not fully solubilized in the oil droplets. The term “pseudo-emulsion” is used as these mixtures are not true emulsions as the solid granules are not fully solubilized into the droplets. The droplets of a pseudo-emulsion have an average droplet diameter of 1 to 20 micrometers, thus being a “solid granule modified macroemulsion”.

Microemulsions are thermodynamically stable dispersions of oil in water, with oil being defined as any water-insoluble liquid. Microemulsion are made by simple mixing of the components. Thus, microemulsions spontaneously form and do not require high shear forces. Unlike macroemulsions, microemulsions do not substantially scatter light. The IUPAC definition of a microemulsion is a “dispersion made of water, oil, and surfactant(s) that is an isotropic and thermodynamically stable system with dispersed domain diameter varying approximately from 1 to 100 nm, usually 10 to 50 nm.” Thus, the droplets of a microemulsion are approximately three orders of magnitude smaller than the droplets of a macroemulsion and are thermodynamically stable.

Nanoemulsions have average droplet diameters from 10 to 125 nanometers, thus being at least an order of magnitude smaller in average droplet diameters than macro- and pseudo-emulsions. Transparent nanoemulsions have average droplet diameters from 10 to 100 nanometers. Nanoemulsions are made with mechanical, high shear forces. While the average droplet diameter of nanoemulsions and microemulsions formally overlap, in practice, the average droplet diameter of nanoemulsions is or becomes larger than those of microemulsions, as lacking the thermodynamic stability of microemulsions, the average droplet diameter of nanoemulsions is forever increasing. It is possible to apply the high shear forces required to form a nanoemulsion to a composition capable of forming a thermodynamically stable microemulsion, however, this will result in a microemulsion as the composition would have “self-assembled” without the high energy forces to form the microemulsion.

Continuous phase means the portion of a microemulsion that carries the droplets that include the substance to be delivered. For example, the modified oil-in-water microemulsions (non-polar droplets in polar continuous phase) addressed herein have oil/alcohol droplets including the alcohol-soluble species to be delivered carried in a polar, “water” continuous phase. While the words “water” and “oil” are used, the “water” can be any liquid that is more polar than the “oil” (such as a polar oil), and the “oil” can be any liquid that is less polar than the “water. Thus, the terms “polar continuous phase” and “water continuous phase” are synonymous, unless water is specifically being discussed as one of the microemulsion components.

Average droplet diameter is determined by dynamic light scattering, sometimes referred to a photon correlation spectroscopy. The determination is made between 20 and 25 degrees Celsius. One example of an instrument suitable for average droplet diameter determination is a Nicomp 380 ZLS particle sizer as available from Particle Sizing Systems, Port Richey, Fla. DLS can determine the diameter of droplets in a liquid by measuring the intensity of light scattered from the droplets to a detector over time. As the droplets move due to Brownian motion the light scattered from two or more droplets constructively or destructively interferes at the detector. By calculating the autocorrelation function of the light intensity and assuming a droplet distribution, it is possible to determine the sizes of droplets from 1 nm to 5 um. The instrument is also capable of measuring the Zeta potential of droplets.

Ingestible means capable of being ingested through the mouth by a living mammal while edible means fit to be eaten, thus in contrast to being unpalatable or poisonous. Edible also means that the composition has less than the permitted amount of viable aerobic microorganisms and meets the American Herbal Products Association (AHPA) guidelines for metals, adulterants, toxins, residual solvents, and pesticides.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these ranges may independently be included in the ranges, and are also encompassed within the invention, subject to any specifically excluded limit in the stated range. Where the stated range includes one or both of the upper and lower limits, ranges excluding either or both of those included limits are also included in the invention.

Unless otherwise indicated, all numbers expressing quantities of ingredients, ratios, and the like used in the specification and claims are to be understood as indicating both the exact values as shown and as being modified by the term “about”. Thus, unless indicated to the contrary, the numerical values of the specification and claims are approximations that may vary depending on the desired properties sought to be obtained and the margin of error in determining the values. At the very least, and not as an attempt to limit the application of the doctrine of equivalents to the scope of the claims, each numerical parameter should at least be construed considering the margin of error, the number of reported significant digits, and by applying ordinary rounding techniques.

While various aspects of the invention are described, it will be apparent to those of ordinary skill in the art that other aspects and implementations are possible within the scope of the invention. Accordingly, the invention is not to be restricted except considering the attached claims and their equivalents.

1. A composition comprising:
an alcohol-soluble species, the alcohol-soluble species comprising dehydroepiandrosterone (DHEA), pregnenolone, and diindolylmethane (DIM); and
a modified oil-in-water microemulsion including a modified oil phase and a modified polar continuous phase,
where the alcohol-soluble species is solubilized in the modified oil phase, the modified oil phase comprising a phospholipid, a polyethylene glycol derivative, an oil, and an alcohol, and
where the modified polar continuous phase comprises a sugar or sugar alcohol and water.

2. The composition of claim 1, where the modified oil-in-water microemulsion is visually clear.

3. The composition of claim 1, where the modified oil-in-water microemulsion is shelf-stable.

4. The composition of claim 1, where the modified oil-in-water microemulsion is ingestible and edible.

5. The composition of claim 1, where the modified oil-in-water microemulsion is configured to provide uptake of the alcohol-soluble species to the bloodstream of a mammal at a therapeutically effective concentration through the oral and gastric mucosa of the mammal.

6. The composition of claim 1, where the composition is configured to provide a human subject a 200 to 500 ug/dL blood concentration of the dehydroepiandrosterone or a metabolite of the dehydroepiandrosterone over a baseline bloodstream concentration within 60-minutes of orally introducing approximately 10 mg of the composition to the human subject.

7. The composition of claim 1, where the composition is configured to orally provide at least 25% by weight of the dehydroepiandrosterone to the bloodstream of a human subject within approximately 180-minutes of the human subject orally ingesting the composition.

8. The composition of claim 1, where the composition is configured to provide at least 14% by weight of the dehydroepiandrosterone to the bloodstream of a human subject within approximately 60-minutes of the human subject orally ingesting the composition.

9. The composition of claim 1, where the modified oil phase is dispersed in the modified polar continuous phase.

10. The composition of claim 9, where droplets of the modified oil phase have an average droplet diameter of 1 to 100 nanometers.

11. The composition of claim 9, where droplets of the modified oil phase have an average droplet diameter of 7 to 30 nanometers.

12. The composition of claim 1, the alcohol-soluble species further comprising testosterone.

13. The composition of claim 1, the alcohol-soluble species further comprising progesterone.

14. The composition of claim 1, the alcohol-soluble species further comprising chrysin.

15. The composition of claim 1, where the alcohol-soluble species comprises a plant sterol.

16. The composition of claim 15, the plant sterol chosen from Tribulus terrestris, yohimbe, and combinations thereof.

17. The composition of claim 1, where the modified oil phase directly solubilizes the dehydroepiandrosterone (DHEA) and the pregnenolone.

18. The composition of claim 17, the modified oil phase further comprising a derivatized hormone.

19. The composition of claim 18, the derivatized hormone chosen from testosterone-propionate, testosterone-cypionate, testosterone-enanthate, testosterone-phenylpropionate, and combinations thereof.

20. The composition of claim 1, the modified oil phase further comprising a cannabis extract.

21. The composition of claim 1, the modified oil phase further comprising a terpene.

22. The composition of claim 1, where the phospholipid is a glycerophospholipid isolated from lecithin.

23. The composition of claim 22, where the phospholipid is chosen from phosphatidylcholine, phosphatidylethanolamine, phosphatidylinositol, ceramide phosphoryl ethanolamine, ceramide phosphoryl choline (SPH), and combinations thereof.

24. The composition of claim 22, where the phospholipid is chosen from phosphatidylcholine, phosphatidylethanolamine, and combinations thereof.

25. The composition of claim 22, where the phospholipid is at least 80% by weight phosphatidylcholine.

26. The composition of claim 1, where the polyethylene glycol derivative is chosen from polyethylene glycol modified vitamin E, polysorbate 40, polysorbate 60, polysorbate 80, and combinations thereof.

27. The composition of claim 26, where the polyethylene glycol modified vitamin E is tocopheryl polyethylene glycol succinate 1000.

28. The composition of claim 1, where the oil is chosen from a medium chain triglyceride, a citrus oil, and combinations thereof.

29. The composition of claim 28, the medium chain triglyceride chosen from caproic acid (hexanoic acid), caprylic acid (octanoic acid), capric acid (decanoic acid), lauric acid (dodecanoic acid), and combinations thereof.

30. The composition of claim 28, the medium chain triglyceride chosen from caprylic acid, capric acid, and combinations thereof.

31. The composition of claim 28, the citrus oil chosen from orange oil, lemon oil, and combinations thereof.

32. The composition of claim 1, where the alcohol is 95% ethanol by weight.

33. The composition of claim 1, the sugar or sugar alcohol chosen from sucrose, cane sugar, pure maple syrup, glycerol, and combinations thereof.

34. The composition of claim 1, where the sugar or sugar alcohol is glycerol.

35. The composition of claim 1, where the alcohol-soluble species comprises from 0.2% to 5% of the composition by weight.

36. The composition of claim 1, where the ratio of the phospholipid, to the oil, to the polyethylene glycol derivative, to the alcohol, to the sugar or sugar alcohol, and to the water is 1:2:0.6-3.3:4:10.5:1-1.6±20% by weight.

37. The composition of claim 1, where the ratio of the phospholipid, to the oil, to the polyethylene glycol derivative, to the alcohol, to the sugar or sugar alcohol, and to the water is 1:2:0.6-3.3:4:10.5:1-1.6±10% by weight.

38. The composition of claim 1, where the ratio of the oil to the alcohol-soluble species is 1:0.02 to 0.3±10% by weight.

39. The composition of claim 1, where the ratio of the oil to the alcohol-soluble species is 1:0.02 to 0.3±5% by weight.

40. The composition of claim 1, where the phospholipid comprises from 3% to 10% of the composition by weight.

41. The composition of claim 1, where the polyethylene glycol derivative comprises from 5% to 14% of the composition by weight.

42. The composition of claim 1, where the ratio of the phospholipid to the polyethylene glycol derivative is 1:0.4 to 1:4 by weight.

43. The composition of claim 1, where the ratio of the phospholipid to the polyethylene glycol derivative is 1:1.6 to 1:4 by weight.

44. The composition of claim 1, where the oil comprises from 5% to 15% of the composition by weight.

45. The composition of claim 1, where the alcohol comprises from 5% to 25% of the composition by weight.

46. The composition of claim 1, where the oil to the alcohol ratio is 1:1.5 to 1:4 by weight.

47. The composition of claim 1, where the sugar or sugar alcohol comprises from 43% to 56% of the composition by weight.

48. The composition of claim 1, where the sugar or sugar alcohol comprises from 48% to 52% of the composition by weight.

49. The composition of claim 1, the modified oil phase further comprising less than 5% by weight of the oil, where the sugar or sugar alcohol comprises from 53% to 63% of the composition by weight.

50. The composition of claim 1, where the water comprises from 2% to 10% of the composition by weight.

51. A method of making a modified oil-in-water microemulsion composition, the method comprising:
combining a phospholipid, a polyethylene glycol derivative, an oil, and an alcohol to form an alcohol-lipid mixture;
combining a sugar or sugar alcohol and water to form a modified polar continuous phase; and
combining an alcohol-soluble species with the alcohol-lipid mixture and the modified polar continuous phase at atmospheric pressure to form the modified oil-in-water microemulsion.

52.-57. (canceled)

58. A method of orally delivering alcohol-soluble species dehydroepiandrosterone and pregnenolone to the bloodstream of a human subject, the method comprising:
introducing a composition comprising the alcohol-soluble species orally to a human subject, the composition comprising:
alcohol-soluble species comprising dehydroepiandrosterone and pregnenolone; and
a modified oil-in-water microemulsion comprising a modified oil phase and a modified polar continuous phase,
where the alcohol-soluble species are solubilized in the modified oil phase, the modified oil phase comprising a phospholipid, a polyethylene glycol derivative, an oil, and an alcohol, and
where the modified polar continuous phase comprises a sugar or sugar alcohol and water; and
delivering the alcohol-soluble species dehydroepiandrosterone and pregnenolone to the bloodstream of the human subject,
where within 60-minutes of the introducing the composition, approximately 2 mL of the composition provides the human subject a blood concentration increase from 200 to 500 ug/dL of the dehydroepiandrosterone or a metabolite of the dehydroepiandrosterone over a baseline pre-introducing bloodstream concentration.

59.-61. (canceled)

62. A method of treating a female human subject in need of hormone replacement therapy with a pulsed dosage regimen, the method comprising:
orally consuming daily for a treatment period of at least two weeks a modified oil-in-water microemulsion, the modified oil-in-water microemulsion comprising:
alcohol-soluble species comprising an effective amount of dehydroepiandrosterone and of pregnenolone; and
a modified oil-in-water microemulsion comprising a modified oil phase and a modified polar continuous phase,
where the alcohol-soluble species are solubilized in the modified oil phase, the modified oil phase comprising a phospholipid, a polyethylene glycol derivative, an oil, and an alcohol, and
where the modified polar continuous phase comprises a sugar or sugar alcohol and water;
at least doubling a baseline dehydroepiandrosterone blood concentration in a bloodstream of the human subject within one hour of the orally consuming to produce an elevated dehydroepiandrosterone blood concentration; and
reducing the elevated dehydroepiandrosterone blood concentration in the bloodstream of the human subject to the baseline dehydroepiandrosterone bloodstream concentration in the bloodstream of the human subject within three hours of the orally consuming.

63.-77. (canceled)
